(19) United States
(12) Patent Application Publication
(10) Pub. No.: US 2006/0177488 A1
(43) Pub. Date: Aug. 10, 2006

(54) Abrasive wipe for treating a surface

(75) Inventors: Pier-Lorenzo Caruso, Pescara (IT); Vincenzo Tomarchio, Brussels (BE); Anne Julienne Alberte Fontaine, Brussels (BE); Giorgio Tinella, Montecchio (IT); Gina Isoldi, Pescara (IT)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH (US)

(21) Appl. No.: 11/348,843

(22) Filed: Feb. 7, 2006

(30) Foreign Application Priority Data
Feb. 7, 2005 (EP) 05075308.6

(51) Int. Cl. A61K 9/70

(52) U.S. Cl. 424/443

(57) ABSTRACT

The present invention relates to a pre-moistened wipe for treating a surface, said pre-moistened wipe comprising: (a) a substrate having a plurality of abrasive means applied thereon, wherein the material forming the abrasive means has a Vickers hardness HV of at least 3 kg/mm² and wherein said abrasive means is a three-dimensional structure having an exposed surface area of at least 0.1 mm²/abrasive means; and (b) a lotion applied to said substrate.

### FIELD OF THE INVENTION

The present invention relates to a pre-moistened abrasive wipe for treating a surface, in particular to a pre-moistened wipe for treating a hard surface. The pre-moistened abrasive wipe incorporates a substrate and a lotion. A pre-moistened wipe according to the present invention was found to exhibit an improved cleaning performance on soils typically found in kitchens and bathrooms, in particular on tough soils such as dried and/or burnt food, while remaining safe to consumers and not damaging to the treated surface, especially to more delicate hard-surfaces such as Plexiglas, linoleum, glass, plastic, plastified wood, or metal.

### BACKGROUND OF THE INVENTION

Wipes for treating surfaces are typically pre-moistened, disposable towelettes which may be utilised in a variety of applications both domestic and industrial and perform a variety of functions. Pre-moistened wipes are typically used to wipe inanimate surfaces, and may provide numerous benefits such as cleaning, cleansing, and disinfecting. Pre-moistened wipes incorporating a cleaning composition are already known in the art.

However, for certain cleaning tasks such as removal of dried and/or burnt food which may typically be found in kitchens, the use of wipes having scrubbing or scouring properties is required. Thus various types of abrasive wipes have been used in the cleaning industry. For example, U.S. Pat. No. 5,213,588 discloses an abrasive wipe consisting of a nonwoven substrate having printed thereon a cured scrubbing bead mixture. Nevertheless, a common and persisting problem with the pre-moistened abrasive wipes for treating hard surfaces known in the art, is that such cleaning wipes which exhibit sufficient abrasiveness for removing tough soils, tend to be overly abrasive to the treated surface and to the user's hand, and thus have limited use when it comes to treat more delicate hard-surfaces such as Plexiglas, linoleum, glass, plastic, plastified wood or metal. In contrast, known abrasive wipes which are suitable for use in the more delicate surfaces exhibit insufficient cleaning performance with respect to tough soils.

Moreover, certain known abrasive pre-moistened wipes make use of abrasive material containing abrasive particles. For example, U.S. Pat. No. 4,078,340 discloses an abrasive pad comprising crimped fibers which are bonded together with a binder that contains finely divided abrasive particles. These types of scrubby wipes are therefore relatively complex and expensive to manufacture.

Thus, the objective of the present invention is to provide a pre-moistened abrasive wipe comprising a substrate and a lotion applied thereon showing an improved cleaning performance benefit on tough soils such as dried and/or burnt food, while remaining safe to the treated surface, especially to more delicate hard-surfaces such as Plexiglas, glazed and non-glazed ceramic tiles, vinyl, no-wax vinyl, linoleum, melamine, glass, plastic, plastified wood or metal.

It has now been found that the above objectives can be met by a pre-moistened abrasive wipe for treating a surface, said pre-moistened abrasive wipe comprising: (a) a substrate having a plurality of abrasive means applied thereon, wherein the material forming the abrasive means has a Vickers hardness HV of at least 3 kg/mm² and wherein said abrasive means is a three-dimensional structure having an exposed surface area of at least 0.2 mm²/abrasive means; and (b) a lotion applied to said substrate.

Advantageously, the abrasive wipes according to the present invention provide a filming/streaking performance benefit (low or substantially no streak- and/or film-formation) on a wide range of stains and surfaces. Another benefit of the present invention is that the excellent cleaning performance is obtained on different types of stains and soils, including greasy stains, as well as particulate stains, especially particulate greasy stains, greasy soap scum and enzymatic stains. A further advantage associated with the abrasive wipes of the present invention is that the pre-moistened abrasive wipes can be easily and inexpensively manufactured.

It is yet another advantage that the pre-moistened abrasive wipes of the present invention when packaged in a box in a stacked configuration, allow an improved dispensing of the wipes out of the packaging box. Incidentally, the pre-moistened abrasive wipes of the present invention are advantageously safe to consumers.

### BACKGROUND ART

WO 02/090483 discloses an impregnated wipe for the cleaning of hard-surfaces, which wipe comprises a substrate having on one side a textured abrasive surface formed from nodules and/or striations of abrasive material applied thereon, the abrasive material having a hardness ranging from 40 to 100 Shore D units.

WO 03/099517 describes a nonwoven composite cleaning pad comprising a first nonwoven layer having an absorbent surface, and a second nonwoven layer laminated to the first layer; and wherein the exposed surface of the second layer is provided with a plurality of abrasive polymer formations.

U.S. application No. 2003/0228813 discloses a scrubbing wipe article comprising a nonwoven substrate and an abrasive resin-based texture layer printed onto the surface of the substrate such that the texture layer extends outwardly beyond the substrate surface.

### SUMMARY OF THE INVENTION

The present invention relates to a pre-moistened wipe for treating a surface, the pre-moistened wipe comprising: (a) a substrate having a plurality of abrasive means applied thereon, wherein the material forming the abrasive means has a Vickers hardness HV of at least 3 kg/mm² and wherein the abrasive means is a three-dimensional structure having an exposed surface area of at least 0.1 mm²/abrasive means; and (b) a lotion applied to the substrate.

In another embodiment, the present invention is directed to a pre-moistened wipe comprising a substrate having a plurality of abrasive means applied thereon, wherein the material forming the abrasive means has a Vickers hardness HV comprised between 3.5 kg/mm² and 20 kg/mm²; and a lotion applied to the substrate.

In a further aspect of the invention, it is provided a process for the manufacture of a pre-moistened abrasive wipe. The process comprises the steps of providing a woven or nonwoven substrate, applying a material having a Vickers hardness HV of at least 3 kg/mm² onto the substrate so as to form a plurality of abrasive means applied thereon, and wherein the abrasive means is a three-dimensional structure having an exposed surface area of at least 0.1 mm²/abrasive means.

### DETAILED DESCRIPTION OF THE INVENTION

Definitions

By ‘substrate’ or ‘wipe’ it is meant any woven or non-woven material formed as a single structure during the manufacturing, or present in the form of two or more material laminates.

By ‘pre-moistened wipe’ it is meant herein a substrate and a lotion as described herein applied to said substrate.

By ‘abrasive’ it is referred to the ability to abrade or remove a relatively small, undesirable item otherwise affixed to a surface as the wipe is moved back and forth over the item.

By ‘abrasive means’ it is meant herein a discrete three-dimensional structure made of an abrasive material.

By ‘exposed surface area’ it is referred to the surface of the material forming the abrasive means which extends outwardly beyond the substrate surface.

In a first embodiment, the present invention relates to a pre-moistened wipe for treating a surface, the pre-moistened wipe comprising: (a) a substrate having a plurality of abrasive means applied thereon, wherein the material forming the abrasive means has a Vickers hardness of at least 3 kg/mm² and wherein the abrasive means is a three-dimensional structure having an exposed surface area of at least 0.1 mm²/abrasive means; and (b) a lotion applied to the substrate.

Substrate

Suitable substrates are well known in the art of wipes and include, but are not limited to, a woven fabric, a knit fabric, a nonwoven fabric, a laminate of a fabric and a polymeric film and combinations thereof. Methods of making such substrate are also well know in the art and are not described in detail herein.

Suitable substrates for use in the present invention are described for example in WO 03/031557 under the paragraph entitled ‘Substrate’ on pages 5 to 12.

Typically, substrates for use herein have homogeneously distributed fibers and are initially dry. Substrates for use herein are impregnated with a lotion at loading factor between 2.0 and 4.40, based on basis weight of the substrate prior to abrasive material application.

By way of example, suitable substrate for use in the present invention may be selected from a hydroentangled 67 g/m² substrate, consisting of 20% polypropylene and 80% PET, and substantially free of binders and latexes. Another example of suitable substrate may be chosen to be a hydroentangled 58 g/m² substrate, consisting of 60% polypropylene and 40% viscose fibers, and substantially free of binders and latexes. Still another example of suitable for use in the context of the present invention is a carded thermobonded 45 g/m² substrate, consisting of 80% polypropylene and 20% viscose fibers, and substantially free of binders and latexes.

The substrate used in the present invention has at least two surfaces, generally a top surface and a bottom surface. The abrasive wipes according to the present invention contain at least one abrasive means on at least one surface of the substrate.

Abrasive Means

The abrasive means according to the present invention are made of any material having the suitable minimum Vickers hardness HV, namely a Vickers hardness HV of at least 3 kg/mm².

Vickers hardness HV is measured at 23° C. according to standard methods ISO 14577-1, ISO 14577-2 and ISO 14577-3. The Vickers hardness HV measurement may be carried out using the Micro-Hardness Tester manufactured by CSM.

The Applicant has surprisingly found that materials having, as a first essential element of the present invention, a minimum Vickers hardness HV of at least 3 kg/mm², preferably at least 3.5 kg/mm², allow obtaining improved cleaning performance on particularly tough soils such as dried and/or burnt food while providing excellent cleaning performance on other regular types of stains and soils, including greasy stains, as well as particulate stains, especially particulate greasy stains, greasy soap scum and enzymatic stains which may typically be found in kitchens and/or bathrooms.

A Vickers hardness HV of at least 3 kg/mm² is found to be necessary to abrade and deteriorate tough soils such as burnt milk stain which Vickers hardness has been determined to be of about 3 kg/mm². The corresponding material exhibits a sufficient hardness to provide an efficient abrasive and destructive action towards burnt milk stain. Such a minimum Vickers hardness will advantageously lead to completely abrade and remove other regular tough soils usually found in kitchens and bathrooms.

In a preferred embodiment, the material forming the abrasive means has a Vickers hardness HV of less than 20 kg/mm². Such a selected range of hardness ensures that commonly known hard-surfaces, and in particular delicate hard-surfaces, are not damaged by the scouring or abrading action of the wipes. Thus, surfaces as fragile as Plexiglas, linoleum, melamine, glass, plastics or plastified wood may be treated with the pre-moistened abrasive wipes according to the present invention without any risk of damaging such delicate surfaces.

It has been indeed determined that by selecting the material forming the abrasive means as having a Vickers hardness inferior to the Vickers hardness of the treated surfaces, the latter remain undamaged after the cleaning action.

Specific examples of the Vickers hardness HV of some common household delicate hard-surfaces are as follows: White Formica (106.5 kg/mm²), Pergo (77.5 kg/mm²), Plexiglas (25.9 kg/mm²), painted wood (6.54 kg/mm²).

The above-mentioned Vickers hardness HV values have been measured according to standard Test Method ISO 14577 using the Micro Hardness Tester manufactured by CSM.

The abrasive wipes of the present invention thus exhibit an improved versatility in terms of the surfaces which may be treated.

In a very preferred execution, the material forming the abrasive means has a Vickers hardness HV comprised between 3 kg/mm² and 25 kg/mm², preferably between 3 kg/mm² and 20 kg/mm², more preferably between 3.5 kg/mm² and 15 kg/mm^{2,} even more preferably between 5 kg/mm² and 15 kg/mm², yet more preferably between 8 kg/mm² and 13 kg/mm², most preferably between 10 kg/mm² and 12 kg/mm².

In a further aspect, the material forming the abrasive means may be free of particulate component, in particular of abrasive particles such as TiO₂, SiO₂ and Al₂O₃. In the context of the present invention, the term particulate component does not encompass beads of polymeric material.

In a preferred execution, the material forming the abrasive means comprises a polymeric material, preferably a thermoplastic polymeric material. More preferably, the material forming the abrasive means is substantially comprised of thermoplastic polymeric material. In an alternative embodiment of the invention, said material forming the abrasive means may comprise commonly known fillers, such as e.g. clay, fumed silica, zeolite, and mixtures thereof.

Suitable thermoplastic polymeric materials for use in the present invention are selected from the group consisting of polyolefins, polystyrenes, polyethers, polyesters, polyamides, vinyl polymers, poly(meth)acrylates, polyurethanes, polycarbonates, mixtures thereof and copolymers thereof.

Specific examples of thermoplastic polymeric materials suitable for use herein include but are not limited to those selected from the group consisting of polyethylene, polypropylene, polyisoprene, polybutylene, atactic polystyrene, polyoxymethylene, polylactic acid, polyethylene terephthalate, polybutyl terephthalate, polyetheramides, polyesteramides, polyamide 6, polyamide 66, polyvinylchloride, polyvinyl alcohol, polyethylene vinylacetate, polymethylmethacrylates, polyurethanes, polycarbonates, mixtures thereof and copolymers thereof.

The abrasive means according to the present invention is a discrete three-dimensional structure having, as a second essential element of the present invention, an exposed surface area of at least 0.1 mm²/abrasive means, preferably of at least 0.2 mm²/abrasive means.

The exposed surface area is a parameter reflecting the three-dimensional morphology of the abrasive means. More specifically, this parameter represents the surface of the material forming the abrasive means which extends outwardly beyond the substrate surface and which will ultimately be the surface of the material forming the abrasive means available for abrasive contact towards the soils to be treated. Incidentally, the exposed surface area of an abrasive means differs from the surface area covered by said abrasive means on the surface of the substrate (i.e. base surface).

The exposed surface area value may be measured using the MicroCT 1172 instrument manufactured by SkyScan or the MicroCT 40 manufactured by Scanco.

The Applicant has surprisingly found that in order to obtain improved cleaning performance a parameter linked to three-dimensional structure of the abrasive means, namely the exposed surface area should be further controlled and optimized. Thus, it has been determined that below an exposed surface area of 0.1 mm²/abrasive means, poorer cleaning performance is obtained in particular with respect to tough soils such as dried and/or burnt food soils.

Without being bound by any theory, it is believed that in the context of cleaning tough soils such as dried and/or burnt food, controlling parameters such as height or diameter of the abrasive means will not as such allow obtaining improved cleaning performance on such soils.

The Applicant has unexpectedly found that the exposed surface area of the abrasive means which corresponds to the surface of the abrasive material in intimate abrading contact with the soils to be cleaned is a critical parameter when trying to further improve cleaning performance on tough soils. It is believed that pre-moistened abrasive wipes according to the present invention, i.e. with the required exposed surface area, provide an overall extended abrading surface available for deteriorating and eliminating tough soils.

Eventually, it is the combination of the material forming the abrasive means (in particular its Vickers hardness HV) with the morphology of the abrasive means (represented by the exposed surface area) which leads to an excellent cleaning performance on tough soils such as burnt milk. It was unexpectedly found that such excellent cleaning performance is obtained without compromising the safety of the treated surfaces.

Preferably, the three-dimensional structure representing the abrasive means has an exposed surface area of less than 10 mm²/abrasive means.

In a preferred embodiment, said three-dimensional structure has an exposed surface area comprised between 0.1 mm²/abrasive means and 5.0 mm²/abrasive means, preferably between 0.2 mm²/abrasive means and 3 mm²/abrasive means, more preferably between 0.5 mm²/abrasive means and 2.0 mm²/abrasive means.

Lotion

Suitable lotions for use in the context of the present invention as well as optional ingredients which may be incorporated in said lotion are described for example in WO 03/031557 under the paragraph entitled ‘Aqueous Composition’ from page 12 to page 36.

Preferably, lotions for use in the present invention are formulated as a liquid composition. A preferred lotion herein is an aqueous composition and therefore, preferably comprises water, more preferably in an amount of from 60% to 99%, even more preferably of from 70% to 98% and most preferably from 80% to 97% by weight of the total lotion composition.

Typically the lotions loaded onto the substrate are made starting from a base composition which preferably comprises 0.05% C12-14 EO21, 8% ethanol, propylene glycol n-butyl ether up to 5%, 0.22% C12-14 sulfobetaine, and acidifying agent up to 2%, and the remainder, perfume, dilution water and anti-foaming, up to 100%, water.

In an independent embodiment herein, the present invention is also directed to a pre-moistened wipe comprising a substrate having a plurality of abrasive means applied thereon, wherein the material forming the abrasive means has a Vickers hardness HV comprised between 3.5 kg/mm² and 20 kg/mm²; and a lotion applied to the substrate.

Such a selected range of hardness ensures that commonly known hard-surfaces, and in particular more delicate hard-surfaces such as Plexiglas, linoleum, melamine, glass, plastics or plastified wood, are not damaged by the scouring or abrading action of the wipes, whilst tough soils such as burnt food are efficiently removed.

In a preferred execution of this other embodiment of the invention, said abrasive means has a Vickers hardness HV comprised between 3.5 kg/mm² and 25 kg/mm², preferably between 3.5 kg/mm² and 20 kg/mm², more preferably between 3.5 kg/mm² and 15 kg/mm² even m ore preferably between 5 kg/mm² and 15 kg/mm², yet more preferably between 8 kg/mm² and 13 kg/mm², most preferably between 10 kg/mm² and 12 kg/mm².

In a further aspect, the material forming the abrasive means may be free of particulate component, in particular of abrasive particles as described herein above.

In a further preferred execution of this other embodiment of the invention, the material forming the abrasive means comprises a polymeric material, preferably a thermoplastic polymeric material. Preferably, the material forming the abrasive means is substantially comprised of thermoplastic polymeric material.

Process for the Manufacture of Pre-Moistened Wipes

In still another embodiment of the invention, it is provided a process for the manufacture of a pre-moistened abrasive wipe, said process comprising the steps of providing a woven or nonwoven substrate; applying a material having a Vickers hardness HV of at least 3 kg/mm² onto said substrate so as to form a plurality of abrasive means applied thereon, wherein said abrasive means is a three-dimensional structure having an exposed surface area of at least 0.1 mm²/abrasive means, preferably of at least 0.2 mm²/abrasive means. Preferably, the material applied onto the substrate has a Vickers hardness HV of at least 3.5 kg/mm².

The abrasive means of the present invention may be applied onto the substrate by any means know in the art, such as roller coating, screen printing, gravure printing, flexographic printing and combinations thereof.

In the context of the present invention, gravure printing is particularly preferred. As being well known in the art, this method will not be described in detail herein.

Regardless the specific technique that was used to apply the material forming the abrasive means onto the substrate, the resulting printed substrate shall be provided with a plurality of abrasive means which are three-dimensional structures having an exposed surface area of at least 0.1 mm²/abrasive means, preferably of at least 0.2 mm²/abrasive means.

The abrasive means are three-dimensional structures which may be of any shape including, but not limited to round, oval, square, triangle, rectangle, rhombus, crescent, star, stripe, grid line, undulating line, circular dot, heart, hexagon, diamond, and combinations thereof.

The abrasive means may be distributed on the substrate surface in a regular manner, such as a plurality of discrete spaced three-dimensional structures, or may be distributed in an irregular manner.

In another execution of the present invention, the plurality of abrasive means are arranged on the substrate surface such as to form a regular or irregular pattern. Virtually, any pattern may be formed on the substrate surface.

In the context of the present invention, it is preferred that the plurality of abrasive means be applied onto said substrate surface so as to achieve a substantial homogeneous distribution over the substrate surface. By ‘substantial homogeneous distribution’ it is meant to reflect that more concentrated portions of abrasive means (i.e. clusters) may be distributed throughout the substrate surface without however leaving said substrate surface with totally uncovered areas while observed on a macroscopic basis.

Typically, the plurality of abrasive means applied on the substrate surface covers an area from 5% to 50%, preferably from 10% to 45%, more preferably from 20% to 40% of said substrate surface.

The overall density of the plurality of abrasive means applied on the substrate surface of the pre-moistened wipe according to the present invention is typically comprised between 10 to 40, preferably between 15 to 30, more preferably between 20 to 25 abrasive means per cm² of the overall substrate surface area.

The specific density of the plurality of abrasive means applied on the substrate surface of the pre-moistened wipe according to the present invention is typically comprised between 5 to 70, preferably between 20 to 50, more preferably between 30 to 40 abrasive means per cm/per each specific cm² of the overall substrate surface area.

According to the present invention, the plurality of abrasive means applied on the substrate surface typically has a basis weight of from 20 g/m² of the abrasive means to 160 g/m² of the abrasive means, preferably from 40 g/m² of the abrasive means to 140 g/m² of the abrasive means, more preferably from 60 g/m of the abrasive means to 120 g/m² of the abrasive means.

In the context of the present invention, it has been surprisingly found that the external surface of three-dimensional structure forming the abrasive means may advantageously be provided with at least one sharp peak.

Without being bound by theory, it is believed that the presence of such peaks will impart a higher degree of abrasiveness to the abrasive means which allows obtaining better cleaning performance in particular on tough soils such as dried and/or burnt food.

Method of Cleaning a Surface

In another embodiment, the present invention encompasses a method of cleaning a surface, preferably a hard surface, comprising the step of contacting, preferably wiping, said surface with a pre-moistened wipe as described herein. In another preferred embodiment of the present application, said process comprises the steps of contacting parts of said surface, more preferably soiled parts of said surface, with said pre-moistened wipe. In yet another preferred embodiment said process, after contacting said surface with said pre-moistened wipe, further comprises the step of imparting mechanical action to said surface using said pre-moistened wipe. By “mechanical action” it is meant herein, agitation of the pre-moistened wipe on the surface, as for example rubbing the surface using the pre-moistened wipe.

By ‘hard-surfaces’, it is meant herein any kind of surfaces typically found in houses like kitchens, bathrooms, or in car interiors or exteriors, e.g., floors, walls, tiles, windows, sinks, showers, shower plastified curtains, wash basins, WCs, dishes, fixtures and fittings and the like made of different materials like ceramic, vinyl, no-wax vinyl, Plexiglas, linoleum, melamine, glass, any plastics, plastified wood, metal or any painted or varnished or sealed surface and the like. Hard-surfaces also include household appliances including, but not limited to, refrigerators, freezers, washing machines, automatic dryers, ovens, microwave ovens, dishwashers and so on.

Packaging Form of the Pre-Moistened Wipes

According to a further embodiment of the present invention, it is provided a packaging form wherein pre-moistened abrasive wipes as described herein are provided in a stacked configuration.

The pre-moistened abrasive wipes according to the present invention may be packaged in a box, preferably in a plastic box.

In a preferred embodiment according to the present invention, the pre-moistened wipes are provided in a stacked configuration, which may comprise any number of wipes.

Typically, the stack comprises from about 2 to about 150, more preferably from about 5 to about 100, most preferably from about 10 to about 60 wipes. Moreover the wipes may be provided in any configuration folded or unfolded. Most preferably, the wipes are stacked in a folded configuration.

The Applicant has surprisingly found that pre-moistened abrasive wipes according to the present invention when packaged in a stacked configuration and in particular when stacked in a folded configuration allow achieving improved dispensing of such pre-moistened wipes from the corresponding package.

Without wishing to be bound by theory, it is believed that improved dispensing and easier grasp of the pre-moistened abrasive wipes of the present invention results from the gap existing between two stacked wipes. The gap is created and facilitated by the existence of a plurality of abrasive means.

Applications

The pre-moistened abrasive wipes according to the present invention may found a wide variety of applications, including but not limited to, hard-surface scrubbing, household cleaning, stain removing, industrial scrubbing. The specific applications will depend upon the desired degree of abrasiveness.

The pre-moistened abrasive wipes of the present invention represent a major improvement over the existing abrasive wipes in terms of soil cleaning performance. This improvement is particularly striking when cleaning performance is evaluated on tough soils such as dried and/or burnt food or greasy kitchen soils. The pre-moistened abrasive wipes herein simultaneously deliver excellent filming/streaking properties on a variety of hard surfaces while remaining safe to consumers and not damaging to the treated surface, especially delicate hard-surface such as Plexiglas, linoleum, melamine, glass, plastic, plastified wood, or metal.

### Test Method

Determination of the Exposed Surface Area

In order to measure the exposed surface of abrasive means extending outwardly beyond the surface of their application substrate, a test method based on high resolution X-ray micro-tomography was used.

This technique reports the X-ray absorption of a sample specimen in the three-dimensional Cartesian coordinates system. The obtained 3D dataset is thus analyzed via Matlab® image processing software application to determine the exposed surface of the 3D material structures extending outwardly beyond the reference level of the application substrate.

Micro-Tomography:

This non-destructive test method is mostly used in the medical and dental field. The sample specimen is irradiated with X-rays. The radiation transmitted through the sample is collected into an X-ray scintillator to transform the X-rays into electromagnetic radiations readable by the CCD elements of an array camera. The obtained 2D image, also called projected image or shadow image, is not sufficient alone to determine independently the X-ray absorption specific for each volume elements (voxels) located along the transmission lines of the X-rays radiated from the source through the sample to the camera. To do so, several projected images taken from different angles are needed to reconstruct the 3D space. The sample specimen is thus rotated (either 180° or 360°) with the smallest possible rotation steps to increase precision. Additional corrections eliminate the positive blur in the back projection process and the distortions induced by the cone beam geometry associated with using a 2D detector.

High resolution micro-tomography is a relatively new field in the area of non-destructive imaging. Current devices are capable of generating projection images in 16 bit depth to discriminate a large number of different X-ray absorption levels. With larger 2D detectors it is possible to reconstruct 200 slices simultaneously with a ratio FOV/resolution>2000 (e.g. 6 μm resolution at 12 mm sample size). 3D datasets are commonly saved as 8 bit images (256 gray levels).

Equipment Needed:

A transfer adhesive (e.g. 1524 grade ex 3M company);
A cutting means able to cut the sample without deformations (e.g. razor blade);
A high resolution desktop X-ray micro-tomography instrument (e.g. Skyscan 1172 or Scanco μCT 40);
A 3D dataset analysis (e.g. a high performance computer to run Matlab®+Image Processing Toolbox).
   Test Procedure:
   1) Sample Preparation

The substrate sample containing the 3D material structures of interest is backed with transfer adhesive. A small specimen 5 mm square (Skyscan) or 12 mm disc (Scanco) is cut from the laminate. Great care must be applied to avoid any laminate stretch or deformation. The backing silicon paper is removed and the sample specimen stuck horizontally onto the rotating cylindrical sample holder of the desktop micro-tomography instrument.

2) Scanning Parameters

For the Skyscan 1172 scanner, the peak voltage of the X-ray source is 100 kVp, the source current is 100 μA, the projection matrix is 1000×524 pixels, the pixel size is 5 μm, the sample rotation cycle is 180°, the rotating step is 0.7°, the beam exposure time at each rotating step is 158 ms, the frame averaging for signal-to-noise reduction is 10. The lowest energy X-rays are not filtered. No random movement to reduce ring artefacts is applied.

For the Scanco μCT 40 scanner, the peak voltage of the X-ray source is 35 kVp, the source current is 110 μA, the projection matrix is 2048×212 pixels, the pixel size is 6 μm, the sample rotation cycle is 360°, the rotating step is 0.18°, the beam exposure time at each rotating step is 250 ms, the frame averaging for signal-to-noise reduction is 5. The lowest energy X-rays are filtered through 300 μm Aluminum. No random movement to reduce ring artefacts is applied.

3) Reconstruction Protocol

The 3D dataset is reconstructed from the projected images obtained at each rotating steps as 1000×1000 pixels (Skyscan) or 2048×2048 pixels (Scanco) matrix per each depth slice, each pixel containing the X-ray absorption in 8 or 16 bit depth format respectively. The pixel size is maintained to 5 μm or 6 μm respectively. Noise smoothing is set as low as possible. Additional post-processing ring artefacts reduction is not required or set to minimum. No X-ray beam hardening correction is required on low X-ray absorbing material or set to minimum.

4) 3D Image Analysis

To determine the surface area of the abrasive means exposed above the surface of the application substrate, we need to isolate the substrate from abrasive means and to define the location of air/substrate interface first, as follows.

a) Dataset Preparation
(i) Loading: The dataset is loaded in Matlab®+Image Processing environment as 16 bit signed integer matrix file (DS1).
(ii) Rescaling: The 3D dataset is re-scaled as 8 bit unsigned integer matrix by assigning the maximum grayscale value just above the X-ray absorbing material extending outwardly beyond the surface of its application substrate (DS2).
(iii) Volume of Interest: A subset containing at least one central 3D structure to analyse is sampled from DS2 (DS3).
(iv) Median Filter: The noise is reduced in the resulting 3D dataset DS3 via median filtering. To do so, the averaging area of the median filter is set half-way below the cross-section of the fibers composing the application substrate. For most samples, 12×12 μm was effective to reduce the noise without affecting the sample specimen (DS4).

The following steps assume a reasonable morphological difference between the 3D structures extending outwardly beyond the surface of their application substrate and the substrate itself, independently from the X-ray absorption.

b) Abrasive Means Identification
(i) Median Filter: The free fibers are removed from the 3D dataset DS3 via another median filtering. To do so, the averaging area of the median filter is set above the cross-section of the fibers composing the application substrate. For most samples, 90×90 μm was effective to remove the free fibers without affecting the morphology of the 3D structures extending outwardly beyond the surface of their application substrate (DS5).
(ii) Binarization: The 3D dataset DS5 is binarized using a gray level threshold calculated via Otsu algorithm: Any voxels with a gray level value above this threshold are set to 1 (material) whilst the other voxels are set to 0 (air) (DS6).
(iii) Depth Map: This 2D image is obtained by projecting the non-zero voxels of the 3D dataset DS6 against a reference x-y plane located below the sample and assigning their distance orthogonal from that plane as pixel value (DM1).

At this point the 3D material structures extending outwardly are clearly identifiable on the depth map.

c) Abrasive Means Exclusion
(i) Binarization: The depth map DM1 is binarized using a gray level threshold calculated via Otsu algorithm: Any pixels with a gray level value above this threshold are set to 1 (3D structures) and the other voxels to 0 (substrate) (DM2).
(ii) Blob Removal: Knots of entangled fibers appear on the binarized depth map DM2 together with the 3D structures to analyse. To remove these knots, a blob analysis is applied on the depth map DM2 setting an area below the minimum expected area of the 3D structures to analyse. For most samples, 0.1 mm² was effective to remove these knots without affecting significantly the 3D abrasive structures to analyse. (DM3).
(iii) Blob Expansion: The remaining blobs located on the binarized depth map DM3 are further expanded (e.g. by 60 μm) to ensure that the remaining area is free from any material belonging to the 3D structures (DM4).
(iv) Exclusion Mask: The depth map DM4 is applied on each horizontal x-y slice of the 3D dataset DS4: Voxels located inside the x-y coordinates of the depth map DM4 are set to 0 whilst the other voxels are unaffected (DS7).

d) Reference Level Determination

The interface between the air and the application substrate is here further defined as reference level. Any material of the 3D structure located above this reference level is exposed and its exposure surface is measurable. The determination of the reference level is obtained from the 3D dataset DS7 containing the sample specimen freed from the 3D structures.
(i) Binarization: The 3D dataset DS7 is binarized using a gray level threshold calculated via Otsu algorithm: Any pixels with a gray level value above this threshold are set to 1 (substrate) and the other voxels to 0 (air) (DS8).
(ii) Reference level Plot: The non-zero voxels of the 3D dataset DS8 are counted for each horizontal x-y plane and reported as histogram of that plane distance from the origin. The higher the distance, the deeper into the substrate.
(iii) Curve Fitting: The aforementioned histogram is fitted with a sigmoid. The reference level is located at the maximum inflexion point in the sigmoid on the axis of the plane distance from the origin.

To note, the image processing procedures mentioned above affect the maximum of the sigmoid but not the position of the reference level. This method is therefore robust to measure the location of the reference level for any substrates.

e) Exposed Surface Area Calculation
(i) Binarization: The 3D dataset DS4 is binarized using a gray level threshold calculated via Otsu algorithm: Any voxels with a gray level value above this threshold are set to 1 (material) whilst the other vozels are set to 0 (air) (DS9).
(ii) Depth Map: This 2D image is obtained by projecting the non-zero voxels of the 3D dataset DS9 above the reference level plane onto it and assigning their distance orthogonal from that plane as pixel value. (DM5).
(iii) Binarization: The depth map DM5 is binarized using a gray level threshold calculated via Otsu algorithm (DM6).
(iv) Blob Removal: Free fibers of the substrate appear on the binarized depth map DM6 together with the 3D structures to analyse. To remove these free fibers, a blob analysis is applied on the binarized depth map DM6 to remove blobs with a diameter just above the cross-section of the fibers composing the application substrate. For most samples, 40 μm was effective to remove the fibers of the application substrate without affecting the 3D structures (DM7).
(v) Inclusion Mask: The depth map DM7 is applied on the depth map DM5 to remove free fibers from the depth map DM5 and include only the peaks of the 3D structures rising above the reference level (DM8).
(vi) Surface Triangulation: The contour points of the rising peaks are defined from the x-y coordinates of the depth map DM8 and the corresponding pixel value as z-coordinate. The contour 3D surface is defined by connecting contour points with any given (x,y) coordinates with neighbouring points at coordinates (x,y+1), (x+1,y) and (x+1,y+1).
(vii) Area calculation: The exposed surface area is obtained by adding each triangle areas of the contour 3D surface.

The obtained exposed surface area in [pixel²] is converted [mm²] knowing the pixel dimensions set by the instrument.

### EXAMPLES

### Example 1

A pre-moistened abrasive wipe according to the present invention was made using a commercially available carded hydro-entangled nonwoven substrate of 58 g/m² basis weight containing 60% polypropylene fibers and 40% absorbent fibers. The applied polymeric material is a blue coloured polypropylene obtained by mixing 1% Macowax blue CW AS78 supplied by Clariant with 99% Moplen HF1005 purchased from Basell. The Vickers hardness HV of the polymeric material is 10 kg/mm². The polymeric material was applied by a gravure printing process as described in EP1262531A1. The dimensions of the engraved cells are 0.14 mm depth and 0.625 mm² base area. The average density is 22 abrasive means per cm² of the overall substrate surface area. The temperature of the gravure roll was set at 165° C. The basis weight of the abrasive means is 93 g/m². The exposed surface area of each abrasive means is 1.07 mm².

### Example 2

A pre-moistened abrasive wipe according to the present invention was made using a commercially available carded hydro-entangled nonwoven substrate of 58 g/m² basis weight containing 60% polypropylene fibers and 40% absorbent fibers. The applied polymeric material is an orange coloured polypropylene obtained by mixing 5% Remafin pe Orange AELF72 supplied by Clariant with 95% Moplen HF1005 purchased from Basell. The Vickers hardness HV of the polymeric material is 10 kg/mm². The polymeric material was applied by a gravure printing process as indicated in Example 1. The dimensions of the engraved cells are 0.14 mm depth and 0.625 mm² base area. The average density is 22 abrasive means per cm² of the overall substrate surface area. The temperature of the gravure roll was set at 180° C. The basis weight of the abrasive means is 105 g/m². The exposed surface area of each abrasive means is 0.56 mm².

### Example 3

A pre-moistened abrasive wipe according to the present invention was made using a commercially available carded hydro-entangled nonwoven substrate of 58 g/m² basis weight containing 60% polypropylene fibers and 40% absorbent fibers. The applied polymeric material is a blue coloured polypropylene obtained by mixing 1% Macowax blue CW AS78 supplied by Clariant with 99% Moplen HF1005 purchased from Basell. The Vickers hardness HV of the polymeric material is 10 kg/mm². The polymeric material was applied by a gravure printing process as indicated in Example 1. The dimensions of the engraved cell are 0.06 mm depth and 0.625 mm² base area. The average density is 31 abrasive means per cm² of the overall substrate surface area. The temperature of the gravure roll was set at 165° C. The basis weight of the abrasive means is 78 g/m². The resulted exposed surface area of each abrasive means is 0.46 mm².

### Example 4

A pre-moistened abrasive wipe according to the present invention was made using a commercially available carded hydro-entangled nonwoven substrate of 58 g/m² basis weight containing 60% polypropylene fibers and 40% absorbent fibers. The applied polymeric material is a blue coloured polypropylene obtained by mixing 1% Macowax blue CW AS78 supplied by Clariant with 99% Moplen HF1005 purchased from Basell. The Vickers hardness HV of the polymeric material is 10 kg/mm². The polymeric material was applied by a gravure printing process as indicated in Example 1. The dimensions of the engraved cell are 0.14 mm depth and 2.598 mm² base area. The average density is 8 abrasive means per cm² of the overall substrate surface area. The temperature of the gravure roll was set at 165° C. The basis weight of the abrasive mean is about 108 g/m². The resulted exposed surface area of each abrasive means is 1.96 mm².

### Example 5

A pre-moistened abrasive wipe according to the present invention was made using a commercially available carded hydro-entangled nonwoven substrate of 58 g/m² basis weight containing 60% polypropylene fibers and 40% absorbent fibers. The applied polymeric material is a blue coloured polypropylene obtained by mixing 1% Macowax blue CW AS78 supplied by Clariant with 82.5% Moplen HF1005 purchased from Basell and 16.5% Vestoplast 703 supplied by Degussa. The Vickers hardness HV of the polymeric material is 6 kg/mm². The polymeric material was applied by a gravure printing process as indicated in Example 1. The dimensions of the engraved cell are 0.10 mm depth and 1.096 mm² base area. The average density is 11 abrasive means per cm² of the overall substrate surface area. The temperature of the gravure roll was set at 160° C. The basis weight of the abrasive mean is 112 g/m². The resulted exposed surface area of each abrasive means is 1.30 mm².

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A pre-moistened abrasive wipe for treating a surface, said pre-moistened wipe comprising:
(a) a substrate having a plurality of abrasive means applied thereon, wherein the material forming the abrasive means has a Vickers hardness HV of at least about 3 kg/mm² and wherein said abrasive means is a three-dimensional structure having an exposed surface area of at least about 0.1 mm²/abrasive means; and
(b) a lotion applied to said substrate.

2. A pre-moistened wipe according to claim 1 wherein said three-dimensional structure has an exposed surface area of at least about 0.2 mm²/abrasive means.

3. A pre-moistened wipe according to claim 1 wherein said abrasive means has a Vickers hardness HV of at least about 3.5 kg/mm².

4. A pre-moistened wipe according to claim 3 wherein said abrasive means has a Vickers hardness HV comprised between about 5 kg/mm² and about 15 kg/mm².

5. A pre-moistened wipe according to claim 1 wherein said material forming the abrasive means comprises a thermoplastic polymeric material selected from the group consisting of polyolefins, polystyrenes, polyethers, polyesters, polyamides, vinyl polymers, poly(meth)acrylates, polyurethanes, polycarbonates, mixtures thereof and copolymers thereof.

6. A pre-moistened wipe according to claim 1 wherein said three-dimensional structure has an exposed surface area comprised between about 0.5 mm²/abrasive means and about 2.0 mm²/abrasive means.

7. A pre-moistened wipe according to claim 1 wherein said plurality of abrasive means applied on the substrate surface has a basis weight of from about 60 g/m² of the abrasive means to about 120 g/m² of the abrasive means.

8. A pre-moistened wipe according to claim 1 wherein said plurality of abrasive means applied on the substrate surface covers an area from about 10% to about 30% of said substrate surface.

9. A pre-moistened wipe according to claim 1 wherein the overall density of said plurality of abrasive means applied on the substrate surface is comprised between about 15 to about 30 abrasive means per cm² of the overall substrate surface area.

10. A pre-moistened wipe according to claim 1 wherein said substrate is composed of a homogeneous blend of synthetic and non-synthetic fibers.

11. A pre-moistened wipe according claim 1 wherein said lotion comprises an aqueous composition.

12. A pre-moistened abrasive wipe for treating a surface, said pre-moistened wipe comprising a substrate having a plurality of abrasive means applied thereon, wherein the material forming the abrasive means has a Vickers hardness HV comprised between about 3.5 kg/mm² and about 20 kg/mm²; and a lotion applied to said substrate.

13. A pre-moistened wipe according to claim 12 wherein said abrasive means has a Vickers hardness HV comprised between about 5 kg/mm² and about 15 kg/mm².

14. A pre-moistened wipe according to claim 12 wherein said material forming the abrasive means comprises a thermoplastic polymeric material.

15. A process for the manufacture of a pre-moistened abrasive wipe, said process comprising the steps of providing a woven or nonwoven substrate; applying a material having a Vickers hardness HV of at least about 3 kg/mm² onto said substrate so as to form a plurality of abrasive means applied thereon, wherein said abrasive means is a three-dimensional structure having an exposed surface area of at least about 0.1 mm²/abrasive means.

16. A process according to claim 15 wherein said three-dimensional structure has an exposed surface area of at least about 0.2 mm²/abrasive means.

17. A process according to claim 15 wherein said material has a Vickers hardness HV of at least about 3.5 kg/mm².

18. A process according to claim 15 wherein said material is applied onto said substrate by a technique selected from the group consisting of roller coating, screen printing, gravure printing, flexographic printing and combinations thereof, preferably by gravure printing.

19. A method of cleaning a surface, comprising the steps of contacting said surface with a pre-moistened wipe according to claim 1, and wiping said surface.
